(19) United States
(12) Patent Application Publication
(10) Pub. No.: US 2020/0303033 A1
(43) Pub. Date: Sep. 24, 2020

(54) SYSTEM AND METHOD FOR DATA CURATION

(71) Applicant: NantOmics, LLC, Culver City, CA (US)

(72) Inventors: Stephen Charles Benz, Santa Cruz, CA (US); Charles Joseph Vaske, Santa Cruz, CA (US); John Thomas Little, Spokane, WA (US); Andrea Preble, Santa Cruz, CA (US)

(21) Appl. No.: 16/356,063

(22) Filed: Mar. 18, 2019

(51) Int. Cl. G16B 20/00; G06F 16/25; G06F 16/22; G06F 16/2457

(52) U.S. Cl. CPC G16B 20/00; G06F 16/24578; G06F 16/22; G06F 16/258

(57) ABSTRACT

A system and method are described for receiving biological process input data from a biological process input data source and transforming the biological process input data into curated data, which may be in a flattened or normalized data space. As an example, a curation server is disclosed with the ability to transform biological process input data from the scientific literature such that the data can be mapped to patient-specific genomic data.

### FIELD OF THE DISCLOSURE

Embodiments of the present disclosure relate to systems and methods for mapping curated data with genomic data.

### BACKGROUND

Genomic data mapping is a time consuming and arduous process; however, the benefits associated with genomic data mapping are undeniable. While genomic data mapping has undergone significant advances in the past year, further improvement can help propel the usefulness of genomic data mapping to real-time or near-real-time patient care.

As a specific problem within the universe of genomic data mapping, there is currently no standardized format for establishing a connection between empirical genomic data. This is primarily due to the fact that there are a myriad of academic papers covering the broad spectrums of: technological areas; publishers; peer review vs. non-peer review, etc. Current methods of reviewing these academic papers do not scale because of various factors. Namely, bias of a researcher and time limits of a researcher are preventing such scaling. Furthermore, because there has been no standardized data format, there has been no central repository or entity that can normalize the data from various sources.

The background includes information that may be useful in understanding the disclosed subject matter. It is not an admission that any of the information provided herein is prior art or applicant admitted prior art, or relevant to the presently claimed inventive subject matter, or that any publication specifically or implicitly referenced is prior art or applicant admitted prior art.

### SUMMARY

It is with respect to the above that embodiments of the present disclosure were contemplated. In particular, embodiments of the present disclosure provide data curation methods and systems. One non-limiting example of such a method includes receiving biological process input data in a first digital data format. The method continues by transforming the biological process input data into curated data, wherein the curated data comprises a second data format in a central dogma of genomics. The method further continues by storing the curated data within a curated database and then receiving patient-specific data. The method then continues by transforming the patient-specific data into the second data format in the central dogma of genomics. The method further continues by mapping the patient-specific data to the curated data within the second data format, determining that at least one instance of data in the patient-specific data matches or nearly matches at least one instance of data in the curated data, and transmitting a report to a client device of a patient or healthcare provider that provides an indication results from the determination that at least one instance of data in the patient-specific data matches or nearly matches at least one instance of data in the curated data.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the inventive subject matter and does not pose a limitation on the scope of the inventive subject matter otherwise claimed. No language in the specification should be construed as indicating any non-claimed element essential to the practice of the inventive subject matter.

Groupings of alternative elements or embodiments of the inventive subject matter disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Various objects, features, aspects and advantages of the inventive subject matter will become more apparent from the following detailed description of preferred embodiments, along with the accompanying drawing figures in which like numerals represent like components.

The phrases “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity may refer to one or more of that entity. As such, the terms “a” (or “an”), “one or more” and “at least one” can be used interchangeably herein. It is also to be noted that the terms “comprising”, “including”, and “having” can be used interchangeably. Also, as used in the description herein, the meaning of “in” includes “in” and “on” unless the context clearly dictates otherwise.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The present disclosure is described in conjunction with the appended figures, which are not necessarily drawn to scale:

FIG. 1 is a block diagram depicting a system in accordance with at least some embodiments of the present disclosure;

FIG. 2 is a block diagram depicting additional details of a system in accordance with at least some embodiments of the present disclosure;

FIG. 3 is a flow chart depicting a method of generating and storing curated data in a database in accordance with at least some embodiments of the present disclosure;

FIG. 4 is a flow chart depicting a method of processing curated data and data models based on confidence levels in accordance with at least some embodiments of the present disclosure;

FIG. 5 is a flow chart depicting a method of processing new inferences regarding drug behavior in accordance with at least some embodiments of the present disclosure;

FIG. 6 is a flow chart depicting a method of resolving conclusion or inference conflicts in accordance with at least some embodiments of the present disclosure;

FIG. 7 is a flow diagram depicting a method of mapping mutations in a patient's genomic data to curated data in accordance with at least some embodiments of the present disclosure;

FIG. 8 is a screen shot illustrating a first graphical user interface in accordance with embodiments of the present disclosure;

FIG. 9 is a screen shot illustrating a second graphical user interface in accordance with embodiments of the present disclosure;

FIG. 10 is a screen shot illustrating a third graphical user interface in accordance with embodiments of the present disclosure;

FIG. 11 is a screen shot illustrating a fourth graphical user interface in accordance with embodiments of the present disclosure;

FIG. 12 is a screen shot illustrating a fifth graphical user interface in accordance with embodiments of the present disclosure; and

FIG. 13 is a screen shot illustrating a sixth graphical user interface in accordance with embodiments of the present disclosure.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

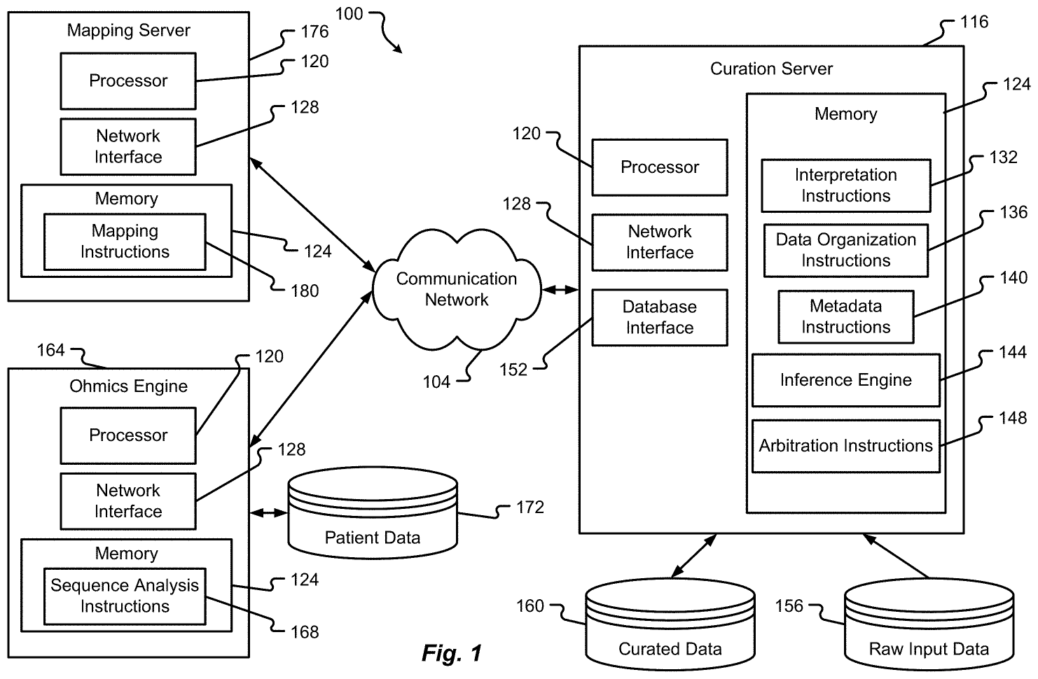

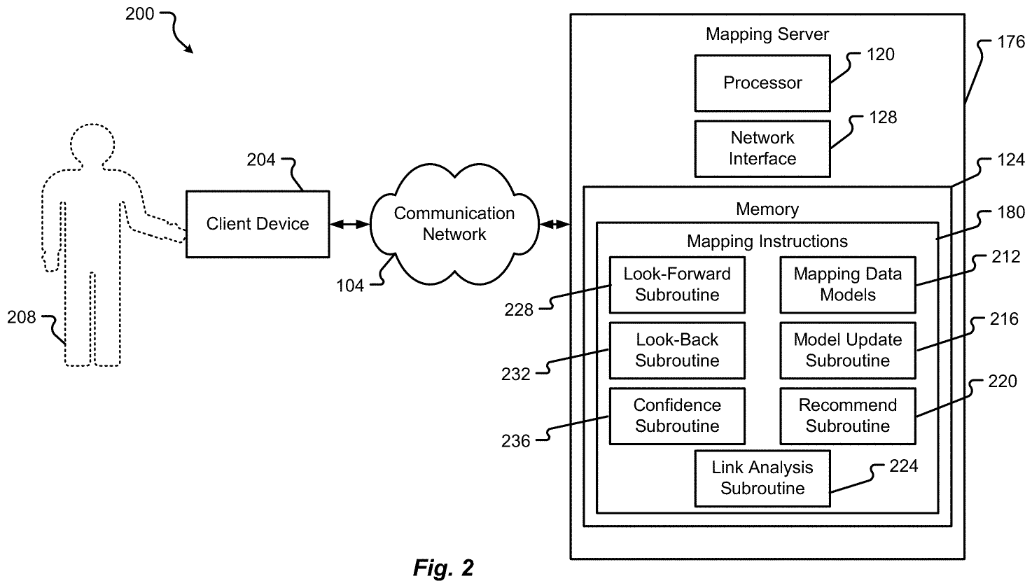

With reference to FIGS. 1 and 2, illustrative systems 100, 200 will be described in accordance with at least some embodiments of the present disclosure. The systems 100, 200, in some embodiments, may include one or more computing devices operating in cooperation with one another to provide data curation and mapping functions. The components of the system 100, 200 may be utilized to facilitate one, some, or all of the methods described herein or portions thereof without departing from the scope of the present disclosure. Furthermore, although particular servers are depicted as including particular components or instruction sets, it should be appreciated that embodiments of the present disclosure are not so limited. For instance, a single server may be provided with all of the instruction sets depicted and described in the servers of FIGS. 1 and 2. Alternatively, different servers may be provided with different instruction sets than those depicted in FIGS. 1 and 2.

The systems 100, 200 are shown to include a communication network 104 that facilitates machine-to-machine communications between one or more servers 116, 164, 176 and/or one or more client devices 204. The system 100 is shown to include a curation server 116, a paradigm (pathway recognition algorithm using data integration on genomic models) server 164, and a mapping server 176. The system 200 shows additional details of the mapping server 176.

The communication network 104 may comprise any type of known communication medium or collection of communication media and may use any type of protocols to transport messages between endpoints. The communication network 104 may include wired and/or wireless communication technologies. The Internet is an example of the communication network 104 that constitutes an Internet Protocol (IP) network consisting of many computers, computing networks, and other communication devices located all over the world, which are connected through many telephone systems and other means. Other examples of the communication network 104 include, without limitation, a standard Plain Old Telephone System (POTS), an Integrated Services Digital Network (ISDN), the Public Switched Telephone Network (PSTN), a Local Area Network (LAN), a Wide Area Network (WAN), a Session Initiation Protocol (SIP) network, a Voice over Internet Protocol (VoIP) network, a cellular network, and any other type of packet-switched or circuit-switched network known in the art. In addition, it can be appreciated that the communication network 104 need not be limited to any one network type, and instead may be comprised of a number of different networks and/or network types. Moreover, the communication network 104 may comprise a number of different communication media such as coaxial cable, copper cable/wire, fiber-optic cable, antennas for transmitting/receiving wireless messages, and combinations thereof.

The client device 204 may correspond to any type of computing resource that includes a processor, computer memory, and a user interface. The client device 204 may also include one or more network interfaces that connect the client device 204 to the communication network 104 and enable the client device 204 to send/receive packets via the communication network 104. Non-limiting examples of client devices 204 include personal computers, laptops, mobile phones, smart phones, tablets, etc. In some embodiments, the client device 204 is configured to be used by and/or carried by a user 208. As will be discussed in further detail herein, the user 208 may utilize a client device 204 to receive and/or view various outputs of the servers 116, 164, 176.

The servers 116, 164, 176 or components thereof may be provided as a single server or in a cloud-computing environment. The curation server 116 may be configured to execute one or multiple different types of instruction sets in connection with processing biological process data received from a biological process input data source 156 and transforming the biological process data into curated data 160 that is useable by the ohmics engine 164 and/or mapping server 176. As will be discussed in further detail herein, the biological process input data received from the biological process input data source 156 may include data relating to (i) the identity of a biological or chemical entity; (ii) the function of a biological or chemical entity; or (iii) the relationship between the function of a biological or chemical entity and another biological or chemical entity. The biological process input data may alternatively, or additionally, include other types of biological data. As will be discussed in further detail herein, the curation server 116 may be configured to process the biological process input data into a normalized (or flattened) data space in which the ohmics engine 164 and/or mapping server 176 are configured to process data. In this way, the curation server 116 helps condition the biological process input data into data that comprises a digestible format for the other servers 164, 176. In some embodiments, the ohmics engine 164 is configured to analyze probabilistic pathway models based on available data. In some embodiments, the curation server 116 may be configured to perform tumor versus normal sequencing.

Biological process input data related to a biological or chemical entity can refer to genes; nucleic acid molecules (DNA or RNA), including sequence information; proteins, including amino acid sequence information and/or three-dimensional structure and/or post-translational modifications; organelles; cells; organs; and organisms. Biological process input data can include information regarding different states of a biological or chemical entity, for example, information regarding an unmodified protein as compared to phosphorylated protein or a free base form of a drug as compared to a salt of the drug. Biological process input data can also include any “omics” data, including genomics, transcriptomics, proteomics, microbiomics, glycomics, lipidomics, or metabolomics.

The curation server 116 is shown to include a processor 120, memory 124, and network interface 128. The mapping server 116 is also shown to include a database interface 152, which may be provided as a physical set of database links and drivers. Alternatively or additionally, the database interface may be provided as one or more instruction sets in memory 124 that enable the processor 120 of the curation server 116 to interact with the databases 156, 160.

These resources of the curation server 116 may enable functionality of the curation server 116 as will be described herein. For instance, the network interface 128 provides the server 116 with the ability to send and receive communication packets over the communication network 104. The network interface 128 may be provided as a network interface card (NIC), a network port, drivers for the same, and the like. Communications between the components of the server 116 and other devices connected to the communication network 104 may all flow through the network interface 128.

The processor 120 may correspond to one or many computer processing devices. For instance, the processor 120 may be provided as silicon, as a Field Programmable Gate Array (FPGA), an Application-Specific Integrated Circuit (ASIC), any other type of Integrated Circuit (IC) chip, a collection of IC chips, or the like. As a more specific example, the processor 120 may be provided as a microprocessor, Central Processing Unit (CPU), or plurality of microprocessors that are configured to execute the instructions sets stored in memory 124. Upon executing the instruction sets stored in memory 124, the processor 120 enables various functions of the curation server 116.

The memory 124 may include any type of computer memory device or collection of computer memory devices. Non-limiting examples of memory 124 include Random Access Memory (RAM), Read Only Memory (ROM), flash memory, Electronically-Erasable Programmable ROM (EEPROM), Dynamic RAM (DRAM), etc. The memory 124 may be configured to store the instruction sets depicted in addition to temporarily storing data for the processor 120 to execute various types of routines or functions. Although not depicted, the memory 124 may include instructions that enable the processor 120 to store or retrieve data from the databases 156, 160. Further still, the memory 124 may include instructions that enable the curation server 116 to provide curated data from the curated database 160 to the ohmics engine 164 and/or mapping server 176 for additional processing.

The illustrative instruction sets that may be stored in memory 124 include, without limitation, interpretation instructions 132, data organization instructions 136, metadata instructions 140, an inference engine 144, and arbitration instructions 148. In some embodiments, the interpretation instructions 124, when processed by the processor 120, enable the curation server 116 to scan and analyze the biological process input data received from the biological process input data source 156 and identify one or more topics, conclusions, recurring words, semantic topics, etc. For example, in the instance of an item (i.e., a datum) of biological process input data being an active pharmaceutical ingredient (API), the curation server 116 may access a data source comprising characteristics or information about the API, such as structural analogs, cell receptor binding partners, metabolic pathways affected by the API, etc. In some embodiments, the interpretation instructions 132 enable the curation server 116 to index the biological process input data and to further assign appropriate tags to the biological process input data. As a specific, but non-limiting example, the biological process input data may correspond to research papers, review papers, research posters, medical publications, clinical trial reports, white papers, or Food and Drug Administration (FDA) or other governing health bodies/agencies reports. Thus, in some embodiments, the biological process input data corresponds to textual data, which may be focused on a particular topic and which may provide one or more conclusions. The interpretation instructions 132 may be configured to automatically scan the text of the biological process input data and extract the topic(s) discussed as well as the conclusions drawn within the biological process input data.

The data organization instructions 136 may be configured to organize the data output by the interpretation instructions 132 for eventual storage as curated data within the curated database 160. For instance, the data organization instructions 136 may enable the server 116 to organize the biological process input data based on the complex data outputs of the interpretation instructions 132 and/or based on inferences drawn by the inference engine 144. In some embodiments, the data organization instructions 136. In some embodiments, the data organization instructions 136 not only organize the biological process input data and index the data based on its organization, but the organization instructions 136 also enable the server 116 to normalize the data to the central dogma of molecular biology. In some embodiments, the data organization instructions 136 may be configured to organize the various data inputs based on a genomics organizational structure. A non-limiting example of a genomics organization structure includes, without limitation, shared/common pathways, cell communication behaviors, and/or cellular network behaviors.

The metadata instructions 140 may be configured to extract metadata from the biological process input data if such metadata already existed within the biological process input data, create additional metadata based on outputs of the data organization instructions 136, and possibly provide inputs to the data organization instructions 136 to assist with the organization of data based, at least in part, on the metadata associated with the biological process input data. In some embodiments, the metadata instructions are configured to generate metadata for the biological process input data that will ultimately match a format of metadata from patient-specific data within the central dogma. Thus, if any metadata will be extracted from or associated with patient-specific data, then corresponding metadata may be extracted from or generated based on the biological process input data. As a non-limiting example, metadata that can be associated with curated data may include patient gender, patient age, article/biological process input data age, article/biological process input data publication date, article/biological process input data author(s) or authorities, treatment information, drug information, combinations thereof, keywords, and the like. Any type of information related to an article or similar type of biological process input data may be formatted into metadata by the metadata instructions 140. In some embodiments, input data could also include “pre-normalized” metadata. For example, papers sometimes include keywords that are used to classify the papers. These keywords could be used as the attributes to map the paper to the central dogma directly. In other embodiments, such metadata might require an additional step of normalizing the data to a common namespace before they can be mapped properly. The main thrust here ensure both the central dogma and the papers can be mapped to each other through a common technology (e.g., a normalized name space).

The inference engine 144, when executed by the processor 120, may enable the server 116 to analyze the biological process input data or curated data to search for inferences that can be made based on the data structure. In some embodiments, the inference engine 144 may be configured to make inferences of drug/treatment efficacy, side-effects, or possibly alternative (yet to be reported) drug/treatment effects. More specifically, the inference engine 144 may be configured to analyze the biological process input data or curated data and generate one or more inferences based on some known biological features from the data and then generate an inference for unknown features to extend one or more known biological features or characteristics. These features can then be mapped to the curated data and, possibly, verified for final mapping or association with the curated data. In some embodiments, an inference may be automatically mapped to or associated with curated data in the event that the inference is made with at least a predetermined confidence score (e.g., a confidence score that meets or exceeds a predetermined threshold). In some embodiments, an inference may require a manual review prior to being mapped to or associated with curated data. For instance, details of the inference generated by the inference engine 144 may be provided to a predetermined user (e.g., a medical professional, a medical researcher, a governmental entity, etc.) for review and approval.

The arbitration instructions 148 may be configured to resolve conflicts between inference rules generated by the inference engine 144 and/or between conclusions drawn between curated data within the database 160. In some embodiments, the arbitration instructions 148 may also enable the curation server 116 to adhere to a predetermined policy or philosophy in connection with resolving such inference conflicts. In some embodiments, these predetermined policies or philosophies may be applied to newly-generated inferences as well as inferenced that were previously generated by the inference engine 144 and stored in connection with curated data. In some embodiments, the arbitration instructions 148 may also be configured to identify and arbitrate between papers or publications having conflicting conclusions. Such conflicting papers can be resolved in a number of different ways. For instance, conflicting papers can be resolved based on a number of citations (e.g., the paper with the greater number of citations may be selected instead of selecting the paper with fewer citations), based on the number of reproduction of results, based on human intervention, or such conflicting papers could be pulled from further analysis. Additional details regarding conflict resolution solutions are described in U.S. Pat. No. 8,510,142, the entire contents of which are hereby incorporated herein by reference. Additional details regarding capabilities of the inference engine 144 are described in U.S. Pat. No. 9,576,242, the entire contents of which are hereby incorporated herein by reference.

Although not depicted, but as will be described in further detail herein, the curation server 116 may also have one or more of its instruction sets (e.g., the inference engine 144) executed as a neural network or similar type of artificial intelligence data structure. Furthermore, these neural networks, such as an intelligent inference engine 144, may be capable of being dynamically trained and updated based on outputs of the curation server 116, based on outputs of the mapping server 176, and/or outputs of the ohmics engine 164. Further still, one or more models used by an intelligent inference engine 144 may be constantly analyzed for possible improvements thereto. Such analysis may be done internally or by an external neural network that is specifically designed to train other neural networks. As another non-limiting example, the data organization instructions 136 may be executed as a neural network whose coefficients between nodes are constantly updated in accordance with desired updates to the data organization for the curated data. For instance, if a particular normalized data space is initially used by the data organization instructions 136, but there is a desire to try a second, different, normalized data space that focuses on different biological information (e.g., shared pathways as compared to cellular communication behaviors), then the data organization instructions 136 may be reconfigured (e.g., offline rather than reconfiguring online with live data) to determine if using a different normalized data space is useful, provides certain benefits, or makes the overall system work less efficiently. If it is determined that the different normalized data space provides an improvement over the original normalized data space, then the data organization instructions 136 may be updated within the curation server 116 to begin applying the new normalized data space to further organizations of the curated data. In some embodiments, one or more neural networks operating as part of the intelligent inference engine 144 may be trained in one or more normalized namespaces. Training of neural networks in a normalize namespace may enable the curation server 116 to generate more deterministic results.

The ohmics engine 164 is shown to include several components that are similar or identical to the curation server 116. For instance, the ohmics engine 164 may include one or more processors 120, a network interface 128, and memory 124. Although not depicted, the ohmics engine 164 may be provided with a database interface 152 to facilitate interactions with a database 172 that stores patient-specific data. In some embodiments, the ohmics engine 164 is provided with sequence analysis instructions 168 that, when executed by the processor 120, enable the ohmics engine 164 to receive and analyze patient-specific data from the database 172. In a more specific but non-limiting example, the sequence analysis instructions 168 may enable the ohmics engine 164 to characterize individual patient tumors and to select therapies based on the identified mutations. Thus, the ohmics engine 164 may be configured to perform matched tumor-normal sequencing analyses to assist in the precise identification and interpretation of somatic and/or germline alterations within a patient's genomic data set. In some embodiments, the sequence analysis instructions 168 may also enable the server 164 to format the patient-specific data from the database 172 based on the central dogma, consistent with a normalize data organization used for the curated data in the curated database 160. In some embodiments, the ohmics engine 164 is configured to receive patient-specific data in the form of one or more of gene expression data from an RNA sequence, CNV profiles from a DNA sequence, protein abundance data from a protein data set, etc. and then prepare one or more interaction pathway data sets that are comparable to the curated data. As an example, pathway activity levels may be comparable to the curated data or a subset thereof within the mapping server 176. For example, methods for using pathway recognition algorithms using data integration on genomic models are disclosed in, for example, U.S. Patent Publication 2012/0041683.

The mapping server 176 is also shown to include a processor 120, memory 124, and network interface 128. In some embodiments, the memory 124 of the mapping server 176 may be provided with mapping instructions 180 that, when executed by the processor 120, enable the mapping server 176 to map patient-specific data received from the ohmics engine 164 with curated data from the curation server 116. In some embodiments, the mapping instructions 180 may include a set of data call instructions that enable the mapping server 176 to call or request instances or discrete sets of patient-specific data from the ohmics engine 164 and call or request related curated data from the curation server 116. The mapping instructions 180 may also receive genomic data from one or both of the ohmics engine 164 and curation server 116 and produce a responder vs. no responder determination from the curated data.

As shown in FIG. 2, the mapping instructions 180 may be provided with a number of subroutines that further enable the functionality of the mapping instructions 180. While the mapping instructions 180 and its subroutines are shown as being provided within the mapping server 176, it should be appreciated that the mapping instructions 180 or subroutines thereof may be provided in the curation server 116 and/or ohmics engine 164 without departing from the scope of the present disclosure. Non-limiting examples of subroutines that may be incorporated within the mapping instructions 180 include mapping data models 212, a model update subroutine 216, a recommendation subroutine 220, a link analysis subroutine 224, a look-forward subroutine 228, a look-back subroutine 232, and a confidence subroutine 236.

The mapping data models 212 may include one or more neural networks or similarly-constructed data-based computer learning models that receive curated data as well as patient-specific data as an input and provide one or more outputs describing a relationship between the curated data and patient-specific data. The mapping data models 212 may be statically defined or may be updated, from time-to-time, with the assistance of the model update subroutine 216. The model update subroutine 216 may be configured to analyze outputs of the mapping data models 212 and determine whether such outputs have been determined an accurate match, a mismatch, a false positive, etc. This information, possibly in the form of a confidence score, may be used by the model update subroutine 216 to determine whether any coefficients within the mapping data models 212 should be updated or whether the models themselves should be updated to achieve a higher accuracy of match between patient-specific data and curated data. A confidence can be measured in a number of different ways. As a non-limiting example, relative distances between patient-specific data and curated data may be measured in one or more high dimensional spaces where the distance is measured as a linear distance in the high dimensional space. In some embodiments, the distance can be measured using a similarity-based mapping method that preserves the original distances between points with respect to any two reference patterns in a special two-dimensional coordinate system, such as a relative distance plane (RDP). A confidence may also be measured using cluster analysis or any other method used to determine similarities between data sets.

The recommendation subroutine 220 may be provided to generate and send one or more recommendations for treatment to a healthcare provider based on a mapping outcome provided by the mapping data models 212. For instance, the mapping data models 212 may indicate that one or more patient-specific data sets are closely related to one or more curated data sets. Information from the curated data set that closely relates to the patient-specific data set may be used to prepare one or more recommendations for treatment to the patient. The recommendation subroutine 220 may even utilize one or more of the link analysis subroutine 224, look-forward subroutine, look-back subroutine 232, and confidence subroutine 236 to assist with the creation of such recommendations and scoring of such recommendations. For instance, the link analysis subroutine 224 can be used to search across pathway interconnects based on known tumor types and known treatments having known outcomes and infer relationships for other tumor types that exhibit similar link behaviors. Pathway interactions can also be used by the look-forward subroutine 228 and look-back subroutine 232 to help determine whether any matches exist between inference structures in the curated data and the patient-specific data. Because the curated data may be formatted based on the central dogma consistent with a format of the patient-specific data, the matching processing performed by the link analysis subroutine 224, with the assistance of the look-forward 228 and look-back subroutines 232 can be performed relatively quickly and efficiently.

The confidence subroutine 246 may enable the mapping instructions 180 to determine a confidence score or index associated with each inference generated by the link analysis subroutine 224 and/or recommendations provided by the recommendation subroutine 220. Such confidence scores may be based on a number of data sources used to generate an inference or recommendation, a degree of proximity between a patient-specific data instance and a curated data instance, a degree of proximity between a patient-specific data instance and data contained in the mapping data models 212, a number of conflicts identified between inferences (e.g., to decrease a particular confidence score), and so on. If a confidence score for a particular inference or recommendation is not at or above a predetermined threshold, then a user 208 may be notified of the deficiency or provided with the actual confidence score to let the user 208 know that an inference or recommendation is not provided with a full level of confidence. As more data is feed into the mapping data models 212, the overall confidence levels may improve or a threshold required for automated functions may increase.

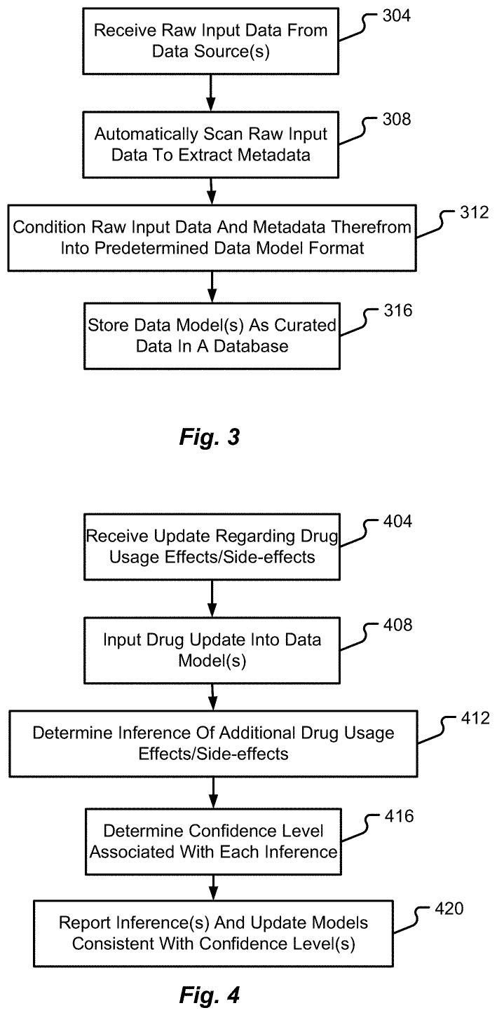

With reference now to FIGS. 3-7, various methods of operating the systems 100, 200 or components therein will be described. It should be appreciated that any of the following methods may be performed in part or in total by any of the components depicted and described in connection with FIG. 1 or 2.

Referring initially to FIG. 3, a method of generating and storing curated data in a database will be described in accordance with at least some embodiments of the present disclosure. The method begins with the curation server 116 receiving biological process input data from one or more biological process input data sources 156 (step 304). In some embodiments, the biological process input data may correspond to textual data received in any digestible format (e.g., a .pdf document, an html document, a word document, a .txt file, a .png file, a .jpeg file, or the like). The method continues with the curation server 116 automatically scanning the biological process input data to extract any metadata therefrom (step 308). In some embodiments, the metadata may be included with the biological process data when received at the curation server 116. For instance, a date of publication of the biological process input data, a date of creation of the biological process input data, author information, and the like may be appended to the biological process input data. Alternatively or additionally, the curation server 116 may scan the content of the biological process input data (e.g., the textual data itself) to extract the appropriate metadata therefrom. For instance, the curation server 116 may perform a keyword or keyphrase search in the text of the document to determine a number of times that words or phrases occur within the document. The curation server 116 may also perform a semantic analysis of the document to determine what, if any, topics are being discussed, what, if any, conclusions were made, what, if any, hypotheses were made, etc. All of the information obtained during the scan of the biological process input data may be used or eventually included as metadata.

The method then continues with the curation server 116 conditioning the biological process input data and the metadata associated therewith into a predetermined data model format (step 312). This predetermined format may be consistent with the central dogma, for example, that can be easily mapped to patient-specific data. In some embodiments, the predetermined format causes the biological process input data to fall into a normalized or flattened data space. Once properly conditioned and organized into the normalized data space, the data may be stored as curated data in the database 160 (step 316). In some embodiments, the curated data may itself be stored as one or more data models. Alternatively or additionally, the curated data may be stored as one or more coefficients within the normalized or flattened data space, thereby making comparisons with patient-specific data more quick and efficient.

With reference now to FIG. 4, a method of processing curated data and data models based on confidence levels will be described in accordance with at least some embodiments of the present disclosure. The method begins with the curation server 116 receiving an update regarding a drug or treatment's usage effects and/or side-effects (step 404). This update may be received by virtue of a new document being added to the biological process input database 156. The new biological process input data may be pushed to the curation server 116 (e.g., by the curation server 116 subscribing to a data feed that automatically pushes such new content to the curation server 116). Alternatively, the new biological process input data may be retrieved by the curation server 116 performing a periodic search against the biological process input database 156 for new entries thereto. In some embodiments, the update may be received in the format of a biological process data input that still requires some amount of analysis by the curation server 116 before being transformed into curated data.

The method continues with the curation server 116 analyzing the update and inputting the update into one or more data models, consistent with a format of the curated data (step 408). For example, the curation server 116 may process the update using the method of FIG. 3 or any other processing method described herein.

The curation server 116 may then apply the inference engine 144 to the newly-created curated data to determine if any inference can be drawn of additional drug/treatment usage effects or side-effects (step 412). In some embodiments, the inference engine 144 may apply a set of inference rules. In some embodiments, the inference engine 144 may even call the look-forward subroutine 228 and/or look-back subroutine 232 of the mapping instructions 180 to determine if, based on the known drug/treatment update, additional drug/treatment updates can be inferred. Even more specifically, the inference engine 144 may analyze other curated data having shared pathways or cell communication behaviors with the update. Based on a proximity between the newly-curated data and previously-existing curated data, the inference engine 144 may draw one or more inferences for additional drug/treatment usage effects and/or side-effects. The inference engine 144 may also determine a confidence level associated with each inference (step 416). The inferences along with their associated confidence levels may be reported to a user 208 and any mapping data models 212 may also be updated (step 420). In some embodiments, the user 208 is notified of the inference(s) by pushing the report to the client device 204.

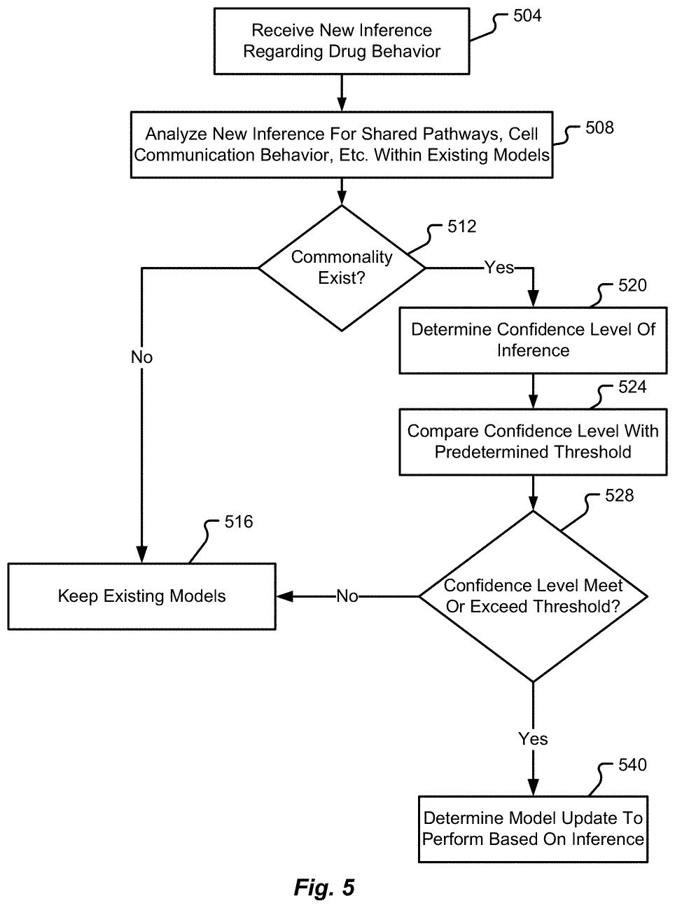

With reference now to FIG. 5, a method of processing new inferences regarding drug behavior will be described in accordance with at least some embodiments of the present disclosure. The method begins by receiving a new inference regarding a drug/treatment behavior (step 504). In some embodiments, the inference received at step 504 may correspond to an output produced by the curation server 116 executing some or all of the method depicted and described in FIG. 4. This output may be maintained within the curation server 116 or may be provided to the mapping server 176 for processing by the mapping instructions 180.

The method continues by analyzing the new inference for shared pathways, cell communication behaviors, etc. within the existing mapping data models 212 (step 508). The method then continues by determining if any commonalities exist between the new inference and the existing models (step 512). In some embodiments, commonalities may be identified by analyzing a relative proximity between the new inference and cause-effect relationships already existing within a data space of the mapping data models. In some embodiments, a feedforward and/or recurrent fuzzy neural network may be used to determine if such commonalities exist. The fuzzy neural network may be configured to learn whether one or more complex temporal sequences exist.

If a commonality does not exist, then the mapping data models 212 are left unchanged (step 516). If, on the other hand, one or more commonalities are determined to exist, then the method continues by determining a confidence level associated with the inference that has the commonality (step 520). The confidence level associated with the inference may be compared with a predetermined threshold value (step 524) to determine if the confidence level meets or exceeds the predetermined threshold (step 528). If the query of step 528 is answered negatively, then the method proceeds to step 516. However, if the query of step 528 is answered affirmatively, then the model(s) 212 may be updated based on the new inference (step 540).

This updating of the models 212 may be performed in an automated fashion in the event that the confidence level meets or exceeds the predetermined threshold value. There may also be a confidence level range in which the updates to the models are suggested to a user 208, but not automatically made. For instance, if the confidence level is less than the predetermined threshold, but greater than a second, lesser, predetermined confidence threshold, then the suggestion may be made to the user 208 to update one or more models or at least consider an update to the one or more models 212. It should be appreciated that the confidence level may be treated as a multi-value parameter or set of parameters rather than treating the confidence level as a single value score. Thus, rather than just passing a predetermined confidence threshold, the multi-value parameter or set of parameters would need to meet more than one criteria to enable an automated update of the models 212.

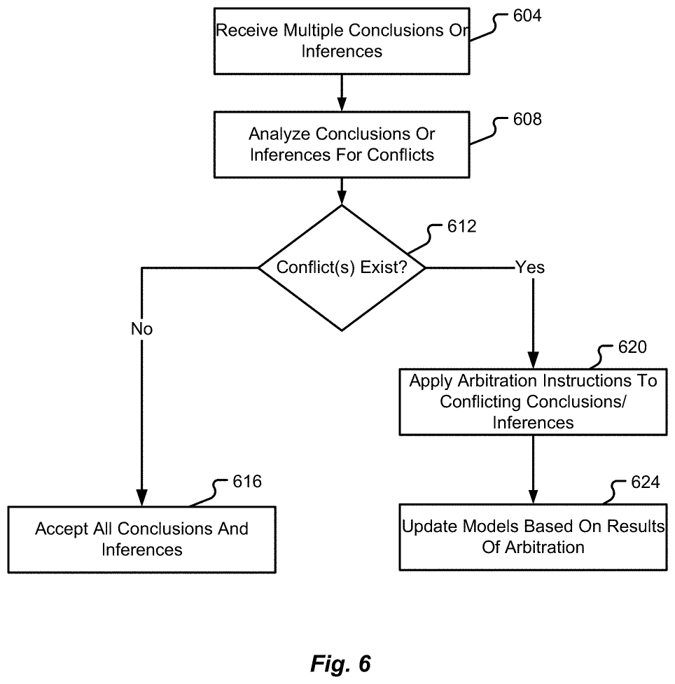

With reference now to FIG. 6, a method of resolving conclusion or inference conflicts will be described in accordance with at least some embodiments of the present disclosure. The method begins by receiving multiple conclusions or inferences within curated data (step 604). The multiple conclusions or inferences may be related to a single instance of curated data or multiple instance of curated data. Furthermore, the conclusions or inferences may correspond to one or more inferences that have been automatically generated by the curation server 116 and/or mapping server 176.

The multiple conclusions or inferences may then be analyzed for conflicts (step 608). For instance, one conclusion or inference may indicate a first predicted outcome/effect/side-effect for a drug or treatment if given to a patient whereas another conclusion or inference may indicate a second predicted outcome/effect/side-effect for the same drug or treatment. The first and second predicted outcomes may correspond to outcomes that are mutually exclusive and incapable of existing simultaneously within a single patient. As a non-limiting example, one conclusion or inference may indicate that a patient will have a positive reaction to a drug or treatment whereas another conclusion or inference may indicate that the patient will not have the positive reaction to the drug or treatment. As another example, one conclusion or inference may indicate that a patient will have a particular side-effect with a drug or treatment whereas another conclusion or inference may indicate that the patient will not have the particular side-effect.

If no conflict exists as determined at step 612, then the multiple conclusions or inference may be accepted without further analysis (step 616). If, however, one or more conflicts exist, then the method may continue by applying the arbitration instructions 148 to the conflicting conclusions or inferences (step 620). In some embodiments, the arbitration instructions 148 may apply a set of inference rules that adhere to a particular policy or philosophy. Examples of such policies may include, without limitation, selecting a conclusion/inference that will have a most negative impact on a patient, selecting a conclusion/inference that has the greatest potential benefit for a patient, selecting a conclusion/inference that has undergone the most peer review or scrutiny, etc. Based on the application of the arbitration instructions 148, the method continues by updating the one or more models 212 and/or modifying instances of curated data consistent with the output of the arbitration instructions 148 (step 624).

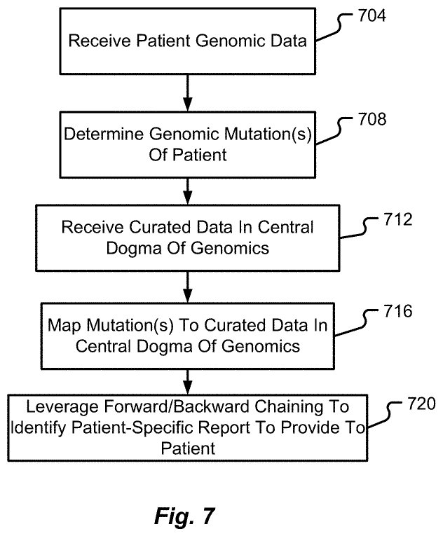

With reference now to FIG. 7, a method of mapping mutations in a patient's genomic data to curated data will be described in accordance with at least some embodiments of the present disclosure. The method begins by receiving patient-specific genomic data (step 704). This particular step may be performed at the ohmics engine 164, which may receive the patient-specific data from the database 172. Alternatively or additionally, this step may be performed by the mapping server 176 receiving the patient-specific data from the ohmics engine 164.

The method continues by identifying one or more genomic mutations within the patient-specific data (step 708). In some embodiments, the patient-specific data may be received in the form of RNA sequence data, DNA sequence data, protein data, or the like, and the mutations may be identified using any known technique. In some embodiments, the mutations may be identified with the sequence analysis instructions 168 or the mutations may already be identified within the patient-specific data.

The method then continues by receiving curated data in the central dogma (step 712). At this point, the mapping server 176 may be in possession of patient-specific data received from the ohmics engine 164 as well as related curated data received from the curation server 116. In some embodiments, the curated data and the patient-specific data may formatted in a normalized data space to facilitate an efficient mapping between the data sets (step 716). The mapping server 176 may further leverage the look-forward subroutine 228 and/or look-backward subroutine 232 to identify whether any possible matches or near matches can be found between the patient-specific data and the curated data. If no such matches or near matches are found, then a report indicating the same may be provided to a user 208 (e.g., a patient or healthcare provider of the patient). However, if one or more matches are identified during the mapping step, then the patient or healthcare provider may be provided with an indication of the matches between the patient-specific data and the curated data (step 720). This information may be provided to the patient or healthcare provider via a client device 204, possibly in the format of an electronic report, which may be transmitted to the user device 204 via the communication network 104.

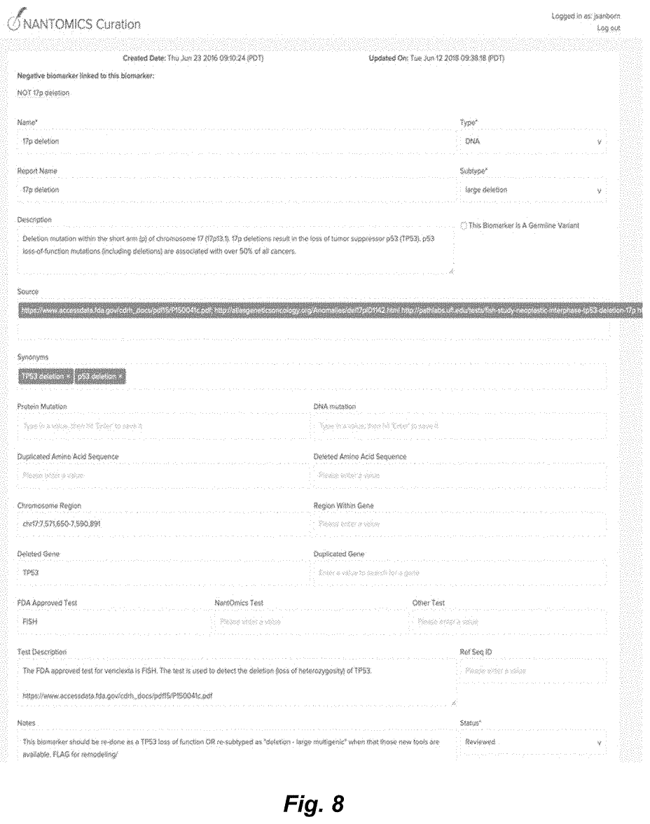

With reference now to FIGS. 8-13, various screen shots of a curation tool enabled by operation of the curation server 116 will be described in accordance with at least some embodiments of the present disclosure. It should be appreciated that the curation tool may present the various graphical user interfaces of FIGS. 8-13 via a display of the client device 204.

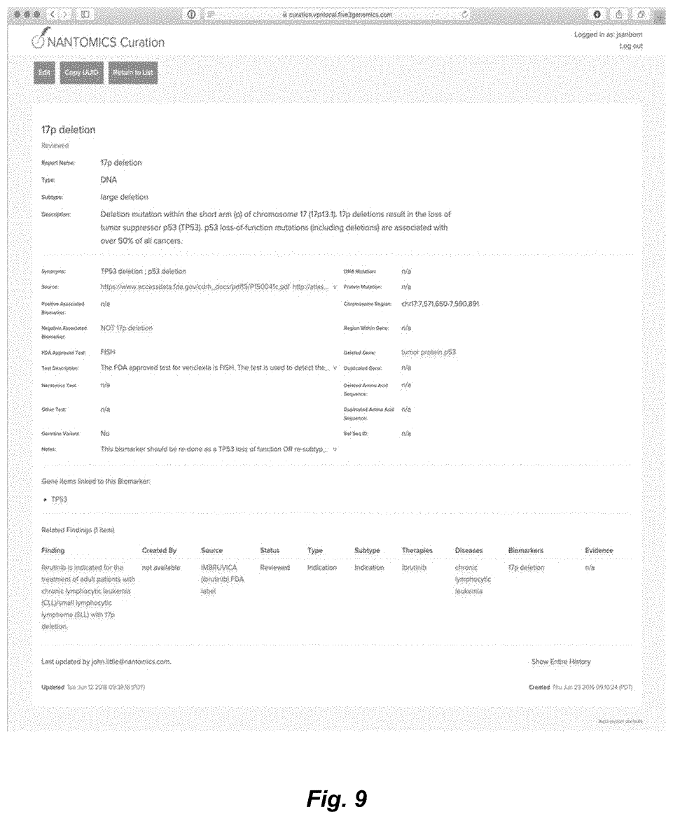

FIG. 8 depicts a graphical user interface presenting a plurality of data fields that can be populated to describe (via data or metadata) curated data. While the graphical user interface of FIG. 8 (and FIGS. 9-13) depicts particular data fields (e.g., biomarker linking fields, name fields, type fields, subtype fields, description fields, source fields, synonym fields, protein mutation fields, DNA mutation fields, duplicated amino acid sequence fields, deleted amino acid sequence fields, chromosome region fields, region within gene fields, deleted gene fields, duplicated gene fields, FDA approval fields, test fields, test description fields, sequence identifier fields, etc.), it should be appreciated that a graphical user interface may include a greater or lesser number of fields or different fields without departing from the scope of the present disclosure.

FIG. 9 depicts a graphical user interface presenting a particular report that includes a report name and various types of data that are related to the report. As shown at the bottom of the graphical user interface, the report may describe findings of the report, sources of the report, status of the report, type and subtype of the report, therapies, diseases, biomarkers, and evidences.

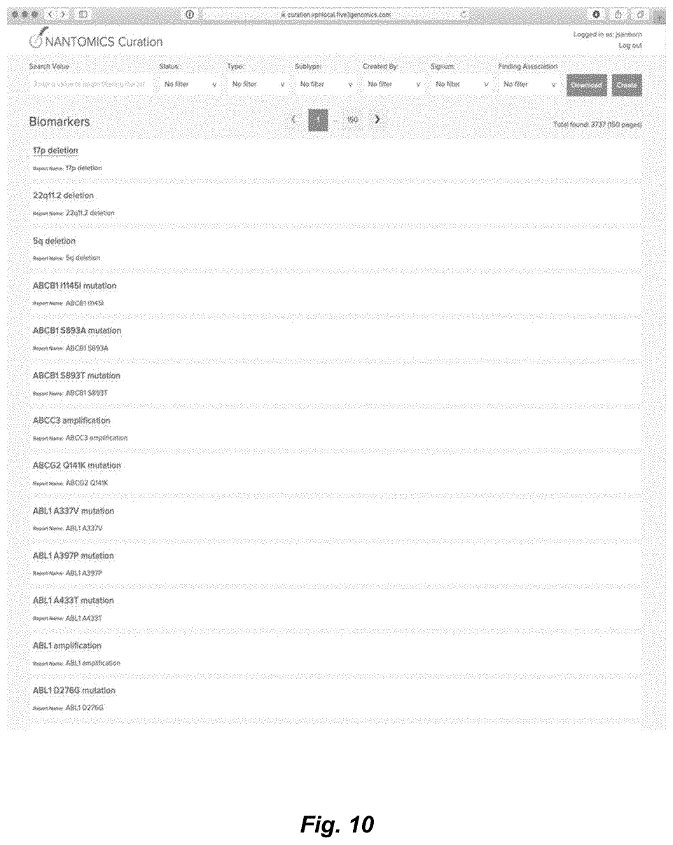

FIG. 10 depicts a graphical user interface depicting details of various biomarkers that may be searched, sorted, and/or filtered through use of the curation tool. In some embodiments, biomarkers may be searched, filtered, or ordered according to status, type, subtype, creator, signum, and finding association information.

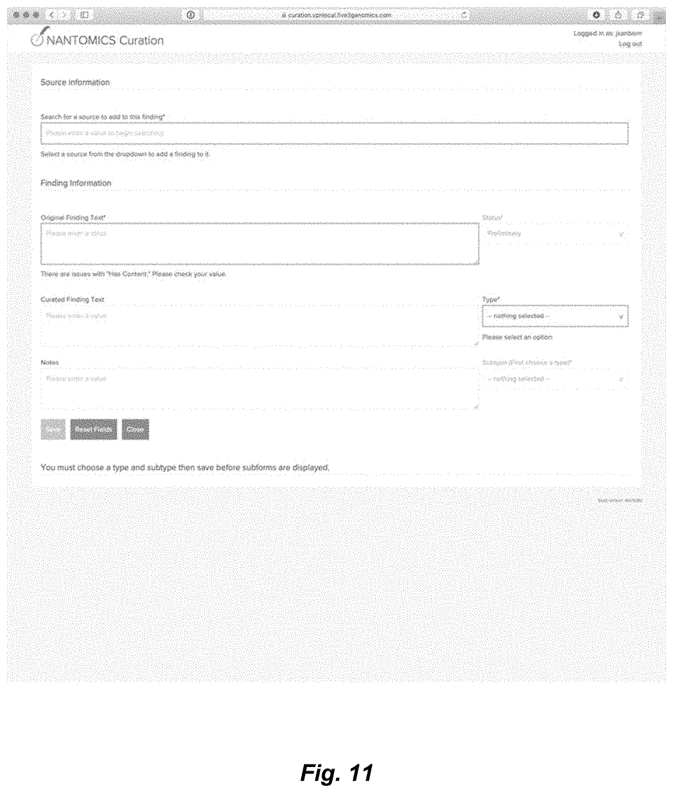

FIG. 11 illustrates a graphical user interface depicting source information and additional fields that may be provided for the source information of curated data. Specifically, but without limiting embodiments of the present disclosure, source information may include sources to add to a finding, original finding information, curated finding information, curator notes, and type/subtype definitions.

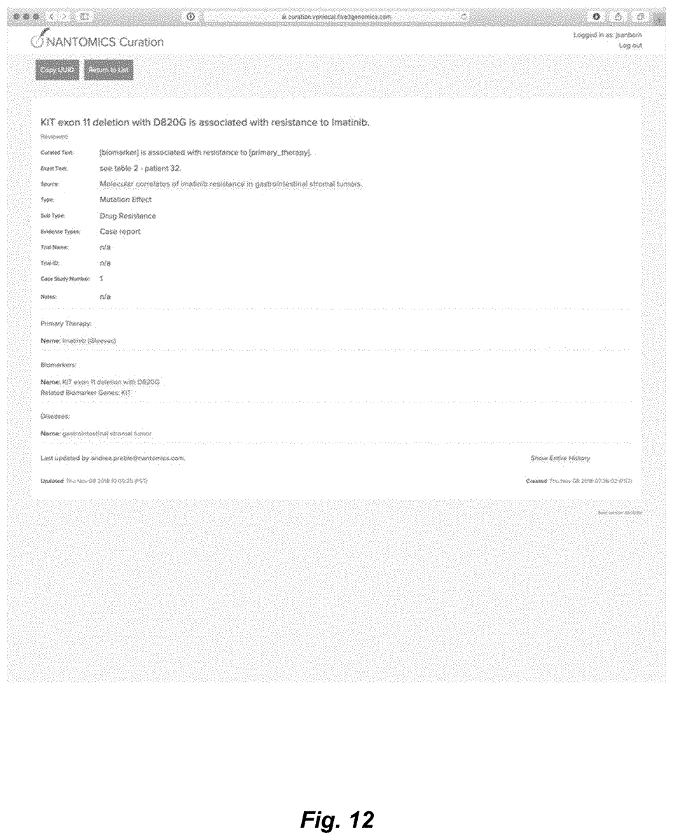

FIG. 12 illustrates a graphical user interface depicting association information between different data sources (e.g., curated data and patient-specific data). The graphical user interface may present association information between curated data and patient-specific data and may further present therapy, biomarker, and disease information associated with one or both data sets.

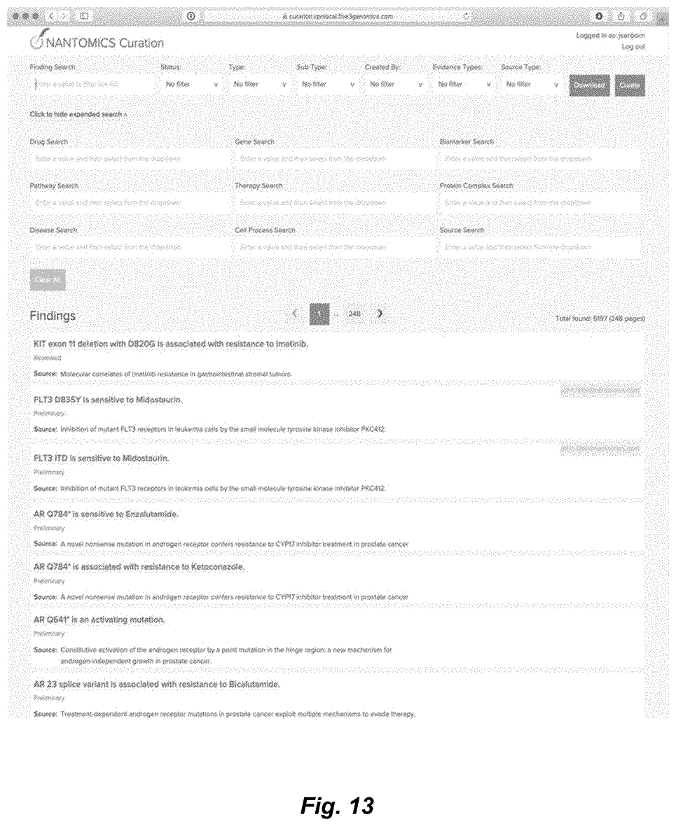

FIG. 13 illustrates a graphical user interface depicting a search or filter tool to search or filter various types of curated data. The search or filter tool may include similar search or filter criteria described above in connection with FIG. 10, but may further include expanded search or filter criteria such as a drug field, a gene field, a biomarker field, a pathway field, a therapy field, a protein complex field, a disease field, a cell process field, and a source field. Curated data may be searched or filtered using one, some, or all of these various data fields.

It should be appreciated that any combination of authentication processes depicted and described herein can be performed without departing from the scope of the present disclosure. Alternatively or additionally, any number of other authentication processes can be developed by combining various portions or sub-steps of the described authentication processes without departing from the scope of the present disclosure.

Specific details were given in the description to provide a thorough understanding of the embodiments. However, it will be understood by one of ordinary skill in the art that the embodiments may be practiced without these specific details. For example, well-known circuits, processes, algorithms, structures, and techniques have been shown without unnecessary detail in order to avoid obscuring the embodiments.

What is claimed is:

1. A computer implemented method, comprising:
receiving biological process input data in a first digital data format;
transforming the biological process input data into curated data in a second digital data format by accessing at least one digital data source comprising at least one characteristic of each datum of the biological process input data and accessing at least one digital data source to extend at least one such characteristic;
storing the curated data within a curated database;
receiving patient-specific digital data;
transforming the patient-specific digital data into the second digital data format;
mapping the patient-specific digital data to the curated data within the second digital data format;
determining that at least one instance of digital data in the patient-specific digital data matches or nearly matches at least one instance of digital data in the curated data; and
transmitting a report to a client device of a patient or healthcare provider that provides an indication results from the determination that at least one instance of data in the patient-specific data matches or nearly matches at least one instance of data in the curated data.

2. The computer implemented method of claim 1, wherein the biological process input data comprises an electronic document.

3. The computer implemented method of claim 2, wherein the biological process input data comprises a text-based document, the method further comprising:
analyzing the text-based document for metadata; and
storing the metadata along with the curated data in the curated database.

4. The computer implemented method of claim 1, further comprising:
determining an inference exists as part of mapping the patient-specific digital data to the curated data;
determining a confidence score for the inference;
comparing the confidence score for the inference with a predetermined confidence threshold; and
automatically accepting the inference as long as the confidence score meets or exceeds the predetermined confidence threshold.

5. The computer implemented method of claim 4, further comprising:
analyzing one or more mapping data models to determine whether the inference impacts the one or more mapping data models;
determining that the inference impacts the one or more mapping data models; and
updating the one or more mapping data models to include the inference.

6. The computer implemented method of claim 5, wherein the one or more mapping data models are updated automatically in response to the confidence score meeting or exceeding the predetermined confidence threshold.

7. The computer implemented method of claim 5, wherein the inference is made by analyzing at least one of a shared pathway interaction or cell communication behavior.

8. The computer implemented method of claim 5, wherein the one or more mapping data models comprise a neural network and wherein updating the one or more mapping data models comprises changing at least one coefficient in the neural network.

9. The computer implemented method of claim 1, wherein the curated data is maintained in a normalized data space.

10. The computer implemented method of claim 1, wherein the biological process input data comprises a research paper, review paper, research poster, medical publication, clinical trial report, white paper, or combinations thereof and wherein the patient-specific data comprises genomic sequence data.

11. A computer implemented method, comprising:
receiving, at a processor, biological process input data in a first data format;
transforming, with the processor, the biological process input data into curated data in a second data format by:
accessing at least one data source comprising at least one characteristic of each datum of the biological process input data;
accessing at least one data source to extend at least one such characteristic; and
storing, with the processor, the curated data within a curated database.

12. The computer implemented method of claim 11, wherein the biological process input data comprises an electronic document.

13. The computer implemented method of claim 12, wherein the biological process input data comprises a text-based document, the method further comprising:
analyzing, with the processor, the text-based document for metadata; and
storing, with the processor, the metadata along with the curated data in the curated database.

14. The computer implemented method of 11, wherein biological process input data comprises data selected from (i) an identity of a biological or chemical entity; (ii) a function of a biological or chemical entity; or (iii) a relationship between the function of a biological or chemical entity and another biological or chemical entity.

15. The computer implemented method of claim 11, wherein extending at least one such characteristic of a datum of the biological process input data is done by inference from the biological process input data, and further comprising:
determining, with the processor, a confidence score for the inference;
comparing, with the processor, the confidence score for the inference with a predetermined confidence threshold; and
automatically accepting, with the processor, the inference as long as the confidence score meets or exceeds the predetermined confidence threshold.

16. The computer implemented method of claim 14, further comprising:
analyzing, with the processor, one or more mapping data models to determine whether the inference impacts the one or more mapping data models;
determining, with the processor, that the inference impacts the one or more mapping data models; and
updating, with the processor, the one or more mapping data models to include the inference.

17. The computer implemented method of claim 15, wherein the one or more mapping data models are updated automatically in response to the confidence score meeting or exceeding the predetermined confidence threshold.

18. The computer implemented method of claim 15, wherein the inference is made by analyzing at least one of a central dogma relationship, shared pathway interaction or cell communication behavior.

19. The computer implemented method of claim 15, wherein the one or more mapping data models comprise a neural network and wherein updating the one or more mapping data models comprises changing at least one coefficient in the neural network.

20. The computer implemented method of claim 11, wherein the curated data is maintained in a normalized data space.

21. The computer implemented method of claim 11, wherein the biological process input data comprises a research paper, review paper, research poster, medical publication, clinical trial report, white paper, or government report.
